(19) United States
(12) Patent Application Publication
(10) Pub. No.: US 2015/0224169 A1
(43) Pub. Date: Aug. 13, 2015

(54) COMPOUNDS AND METHODS FOR SELECTIVELY TARGETING CANCER STEM CELLS

(71) Applicant: McMaster University, Hamilton (CA)

(72) Inventors: Mickie Bhatia, Hamilton (CA); Tony Collins, Hamilton (CA)

(21) Appl. No.: 14/426,269

(22) PCT Filed: Sep. 6, 2013
(86) PCT No.: PCT/CA2013/050688

§ 371 Date: Mar. 5, 2015

Related U.S. Application Data
(60) Provisional application No. 61/697,573, filed on Sep. 6, 2012.

(51) Int. Cl. A61K 38/12; A61K 31/519; C12N 5/09; A61K 31/7048

(52) U.S. Cl. CPC A61K 38/12; A61K 31/7048; A61K 31/519; C12N 5/0693; C12N 5/0694; C12N 2506/30; C12N 2501/40

(57) ABSTRACT

Described are compounds and methods useful for selectively targeting cancer stem cells. The compounds preferentially induce differentiation and/or reduce the proliferation of cancer stem cells relative to normal stem cells. Compounds useful for selectively targeting cancer stem cells include polyene macrolides such as Nystatin or Amphotericin B, analogs thereof and pharmaceutically acceptable salts thereof.

### RELATED APPLICATIONS

This application claims priority to U.S. Provisional Patent Application No. 61/697,573 filed on Sep. 6, 2012, the contents of which are hereby incorporated by reference in their entirety.

### FIELD OF THE DISCLOSURE

The present disclosure relates to cancer stem cells and particularly to compounds and methods for selectively reducing the proliferation of cancer stem cells.

### BACKGROUND OF THE DISCLOSURE

Increasing evidence suggests that cancer/tumor development is due to a rare population of cells, termed cancer stem cells (CSCs) (Dick, 2009; Jordan, 2009; Reya et al., 2001) that are uniquely able to initiate and sustain disease. In addition, experimental evidence indicates that conventional chemotherapeutics, characterized by their ability to inhibit cell proliferation of cancer cell lines (Shoemaker, 2006) or reduce tumor burden in murine models (Frese and Tuveson, 2007), are ineffective against human CSCs (Guan et al., 2003; Li et al., 2008). This resistance to chemotherapeutics is coupled with indiscriminate cytotoxicity by compounds that often affect healthy stem and progenitor cells, leading to dose restriction and necessitating supportive treatment (Smith et al., 2006). Recent examples include selective induction of apoptosis (Gupta et al., 2009; Raj et al., 2011) that remains to be tested in normal stem cells (SCs) and in the human system. Accordingly, the identification of agents that target CSCs alone is now critical to provide truly selective anti-cancer drugs for pre-clinical testing.

Normal and neoplastic stem cells are functionally defined by a tightly controlled equilibrium between self-renewal vs. differentiation potential. In the case of CSCs, this equilibrium shifts towards enhanced self-renewal and survival leading to limited differentiation capacity that eventually allows for tumor growth. In contrast to direct toxic effects that equally affect normal SCs, an alternative approach to eradicate CSCs is by modification of this equilibrium in favor of differentiation in an effort to exhaust the CSC population. The identification of molecules that selectively target somatic CSCs while sparing healthy SC capacity would therefore be useful for the development of novel therapeutic treatments to selectively target human CSCs.

### SUMMARY OF THE DISCLOSURE

In one aspect of the disclosure, compounds which preferentially induce the differentiation of cancer stem cells or reduce the proliferation of cancer stem cells relative to normal stem cells are provided. In one embodiment the compounds preferentially induce the differentiation of cancer stem cells relative to normal stem cells. In one embodiment, the compounds preferentially reduce the proliferation of cancer stem cells relative to normal stem cells. As shown in Example 1, each of the compounds provided herein has been identified using a screening assay for identifying and validating compounds which are selective for variant neoplastic stem cells relative to normal stem cells. In one embodiment, the compounds disclosed herein include Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton, as well as pharmaceutically acceptable salts and variant forms thereof. In one aspect of the disclosure, the compound is Triamterene or colistin sulfate, analogs thereof or pharmaceutically acceptable salts thereof. Furthermore, as shown in Example 4, the polyene macrolide Nystatin reduces the proliferation of leukemic cancer cells without affecting hematopoietic stem cell proliferation. In one aspect of the disclosure, the compound is therefore a polyene macrolide selected from Nystatin, Amphotericin B, analogs thereof and pharmaceutically acceptable salts thereof.

Accordingly, in one embodiment, there is provided a method of reducing the proliferation of cancer stem cells comprising contacting the cancer stem cells with a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton. In one embodiment, there is provided a method of reducing the proliferation of cancer stem cells comprising contacting the cancer stem cells with a polyene macrolide. In one embodiment, the polyene macrolide is selected from Nystatin and Amphotericin B. In one embodiment, the polyene macrolide is an analog or pharmaceutically acceptable salt of Nystatin or Amphotericin B. In one embodiment, there is provided a method of preferentially inducing the differentiation of cancer stem cells comprising contacting the cancer stem cells with a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton. In one embodiment, there is provided a method of preferentially inducing the differentiation of cancer stem cells comprising contacting the cancer stem cells with a polyene macrolide. In one embodiment, the polyene macrolide is selected from Nystatin, Amphotericin B, analogs thereof, and pharmaceutically acceptable salts thereof.

In one embodiment, the compounds described herein preferentially induce the differentiation of cancer stem cells relative to normal stem cells. For example, in one embodiment the compounds described herein preferentially induce the differentiation of neoplastic variant stem cells as compared to normal stem cells such as H9 cells. In one embodiment, the compounds disclosed herein preferentially kill cancer stem cells relative to normal stem cells. In one embodiment, the compound is Triamterene, an analog thereof or pharmaceutically acceptable salt thereof. In one embodiment, the compound is colistin sulfate, an analog thereof pharmaceutically acceptable salt thereof. In one embodiment, the compound is Nystatin, Amphotericin B, an analog thereof, or a pharmaceutically acceptable salt thereof

Optionally, the cancer stem cells may be in vitro, in vivo or ex vivo. In one embodiment, the cancer stem cells are in a subject with cancer or suspected of having cancer. In one embodiment, the subject is in remission. In one embodiment, the compounds described herein are useful for treating a subject with cancer or suspected of having cancer. Also provided are methods for the treatment of cancer in a subject in need thereof, comprising administering to the subject a compound described herein, such as a polyene macrolide. In one embodiment, the compound is a polyene macrolide selected from Nystatin, Amphotericin B, analogs thereof and pharmaceutically acceptable salts thereof. In one embodiment, the compound is Triamterene, an analog thereof or pharmaceutically acceptable salt thereof. In one embodiment, the compound is colistin sulfate, an analog thereof pharmaceutically acceptable salt thereof.

Also provided is the use of a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton for preferentially inducing the differentiation of cancer stem cells relative to normal stem cells or reducing the proliferation of cancer stem cells relative to normal stem cells. In one embodiment, there is provided the use of a polyene macrolide for preferentially inducing the differentiation of cancer stem cells relative to normal stem cells or reducing the proliferation of cancer stem cells relative to normal stem cells. In one embodiment, the polyene macrolide is selected from Nystatin, Amphotericin B, analogs thereof and pharmaceutically acceptable salts thereof. In one embodiment, the compound is Triamterene, an analog thereof or pharmaceutically acceptable salt thereof. In one embodiment, the compound is colistin sulfate, an analog thereof pharmaceutically acceptable salt thereof.

Also provided in the use of a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton for the treatment of cancer. In one embodiment, there is provided the use of a polyene macrolide for the treatment of cancer. In one embodiment, the polyene macrolide is selected from Nystatin, Amphotericin B, analogs thereof and pharmaceutically acceptable salts thereof. In one embodiment, the compound is Triamterene, an analog thereof or pharmaceutically acceptable salt thereof. In one embodiment, the compound is colistin sulfate, an analog thereof or a pharmaceutically acceptable salt thereof. In one embodiment, the compound preferentially induces the differentiation of cancer stem cells relative to normal stem cells. In one embodiment, the compound reduces the proliferation of cancer stem cells relative to normal stem cells, such as H9 cells or hematopoietic stem cells. In one embodiment, the cancer is leukemia, optionally acute myeloid leukemia (AML).

Also provided is a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton for use in the treatment of cancer. In one embodiment, there is provided is a polyene macrolide for use in the treatment of cancer. In one embodiment, the polyene macrolide is selected from Nystatin, Amphotericin B, analogs thereof and pharmaceutically acceptable salts thereof. In one embodiment, the compound is Triamterene, an analog thereof or pharmaceutically acceptable salt thereof. In one embodiment, the compound is colistin sulfate, an analog thereof or pharmaceutically acceptable salt thereof. In one embodiment, the cancer is leukemia, optionally AML.

Also provided is the use of a compound described herein for the manufacture of a medicament or a pharmaceutical composition for the treatment of cancer.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

One or more embodiments of the disclosure will now be described in relation to the drawings in which:

FIG. 1 shows the workflow for the screening assay for identifying and validating compounds that selectively target cancer stem cells but not normal stem cells (H9).

FIG. 2 shows a bar chart identifying compounds with the highest selectivity-activity ratios for cancer stem cells (v1O4) relative to normal stem cells (H9). The selective-activity potency ratio is determined by EC50_{H9}÷EC50_{v1O4}.

FIG. 3A shows dose-response curves of selective-activity compounds that exhibit selectivity at 10 μM. FIG. 3B shows dose-response curves of selective-activity compounds that do not necessarily exhibit selectivity at 10 μM but are nevertheless selective at other concentrations. Cell counts are normalized to untreated controls. Dashed line is 10 μM concentration. Screening compounds at a plurality of test concentrations is therefore useful for identifying compounds that are selective for anti-cancer agents.

FIG. 4 shows that only a small subset (5%) of known anti-cancer drugs from screening libraries show selective activity against variant neoplastic stem cells.

FIG. 5 shows a plot of the percentage of v1O4 or H9 cells that stain positive for p53 after treatment with high selective-activity compounds (grey). High levels of p53 indicate activation of the p53-dependent stress response. The black dots represent p53 levels of v1O4 and H9 cells treated with thioridazine and thio-structure-like compounds. The thio-structure-like compounds shown in this figure include: proclorperazine, trifluoperazine, fluphenazine and perphenazine. High selectivity compounds have varying degrees of p53 stress response activation activity.

FIG. 6A shows the quantification of CFUs and blast-CFUs generated from cord blood and AML cells following treatment with Nystatin and Cytarabine (AraC). Values were normalized to control samples treated with 0.1% DMSO. Dotted line indicates DMSO control at 1. Each bar represents n=6 individual samples, mean±SEM. *P<0.05, **P<0.01, ***P<0.001 (comparing normalized counts). # P<0.05, ## P<0.01 (compared to DMSO absolute count). FIG. 6B shows the ratio of normalized cord blood CFUs per AML-blast CFUs after treatment with the same concentrations Nystatin or AraC. * represents statistically significant difference between normalized number of CFUs from cord blood and normalized number of blast CFUs from AML for the indicated treatment group (P<0.05, t-test).

FIG. 7 shows the chemical structures of nystatin (A), amphotericin B (B).

### DETAILED DESCRIPTION

### I. Definitions

As used herein, the term “cancer” refers to one of a group of diseases caused by the uncontrolled, abnormal growth of cells that can spread to adjoining tissues or other parts of the body. Cancer cells can form a solid tumor, in which the cancer cells are massed together, or exist as dispersed cells, as in leukemia.

The term “leukemia” as used herein refers to any cancer involving the progressive proliferation of abnormal leukocytes found in hemopoietic tissues, other organs and usually in the blood in increased numbers. “Leukemic cells” refers to leukocytes characterized by an increased abnormal proliferation of cells. Leukemic cells may be obtained from a subject diagnosed with leukemia.

The term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells.

As used herein the term “cancer stem cell” refers to a cell that is capable of both self-renewal and differentiating into the lineages of cancer cells that comprise a tumor or hematological malignancy. Cancer stem cells are uniquely able to initiate and sustain cancer. Variant neoplastic stem cells are cells which exhibit the properties of cancer stem cells and are useful in the screening methods described herein for identifying and/or validating anti-cancer stem cell agents. Variant neoplastic stem cells are described in Example 1, as well as in Werbowetski-Ogilvie et al., (2009) and Sachlos et al., (2012) both hereby incorporated by reference in their entirety.

As used herein, a “normal stem cell” is a stem cell that is not a cancer stem cell or a variant neoplastic stem cell. Examples of “normal” stem cells include pluripotent stem cells, embryonic stem cells such as H9 stem cells and hematopoietic stem cells. Other “normal” stem cells include cells found in lineage depleted cord blood which represents a population of normal hematopoietic progenitor cells and normal hematopoietic stem cells.

As used herein, “reducing the proliferation of a cancer stem cell” refers to a reduction in the number of cells that arise from a cancer stem cell as a result of cell growth or cell division and includes cell death or differentiation of a cancer stem cell. The term “cell death” or “killing a cancer stem cell” as used herein includes all forms of cell death including necrosis and apoptosis. As used herein “differentiation of a cancer stem cell” refers to the process by which a cancer stem cell loses the capacity to self-renew and cause the lineages of cancer cells that comprise a tumor or hematological malignancy.

As used herein, the phrase “effective amount” or “therapeutically effective amount” means an amount effective, at dosages and for periods of time necessary to achieve the desired result. For example in the context or treating cancer, an effective amount is an amount that for example induces remission, reduces tumor burden, and/or prevents tumor spread or growth of leukemic cells compared to the response obtained without administration of the compound. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “pharmaceutically acceptable” means compatible with the treatment of animals, in particular, humans.

The term “pharmaceutically acceptable salt” means an acid addition salt or a base addition salt which is suitable for, or compatible with, the treatment of subjects. The embodiments described herein include pharmaceutically acceptable salts of a polyene macrolide such as nystatin, and amphotericin B, or analogs thereof.

An “acid addition salt which is suitable for, or compatible with, the treatment of subjects” is any non-toxic organic or inorganic salt of any basic compound. Basic compounds that form an acid addition salt include, for example, compounds comprising an amine group. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

A “base addition salt which is suitable for, or compatible with, the treatment of subjects” is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid in a suitable solvent and the formed salt is isolated by filtration, extraction or any other suitable method.

The term “subject” as used herein includes all members of the animal kingdom including mammals, and suitably refers to humans. Optionally, the term “subject” includes mammals that have been diagnosed with cancer or are in remission.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. In one embodiment, treatment methods comprise administering to a subject a therapeutically effective amount of a compound as described herein and optionally consists of a single administration, or alternatively comprises a series of administrations.

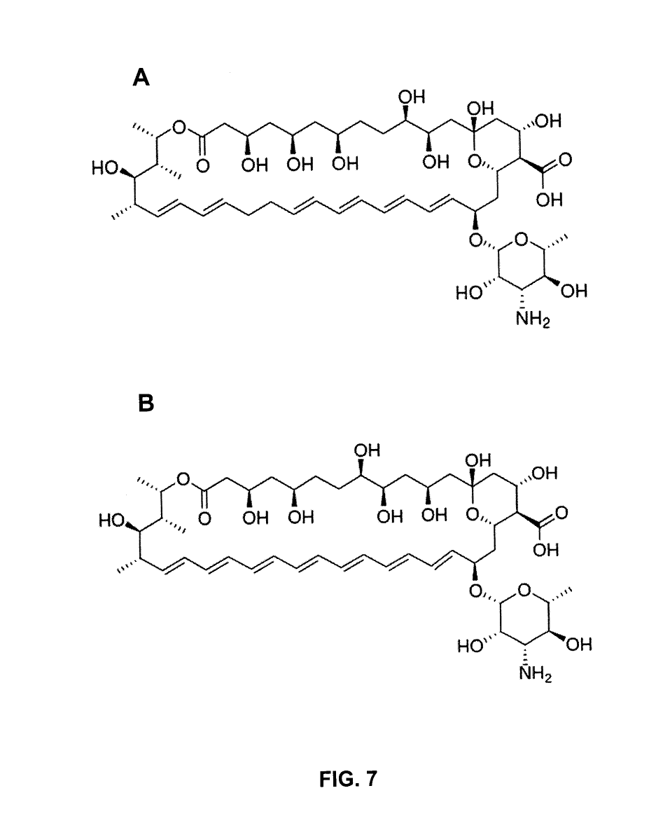

As used herein, “polyene macrolide” refers to an organic compound characterized by the presence of a macrocyclic lactone ring and one or more sequences of alternating double and single carbon-carbon bonds. Polyene macrolides are commonly used as antifungal agents and believed to interact with membrane sterols resulting in the formation of hydrophilic channels through which small molecules and ions can leak out of the cell. In one embodiment, polyene macrolides bind sterols such as cholesterol or ergosterol in cell membranes. Examples of polyene macrolides include nystatin produced by Streptomyces noursei ATCC 11455, as well as amphotericin B. Polyene macrolides such as nystatin are also known to inhibit specific endocytic pathways in non-fungal cells that are mediated by cholesterol rich regions of the plasma membrane called caveolae or lipid rafts (See e.g. Chen et al., 2011). The chemical structures of nystatin and amphotericin B are each shown in FIG. 7. Nystatin analogs include those compounds described by Brautaset et al. (2008) that share structural and functional properties with nystatin. A person skilled in the art would also readily be able to identify analogs and pharmaceutically acceptable salts of the polyene macrolides described herein.

### II. Methods and Uses

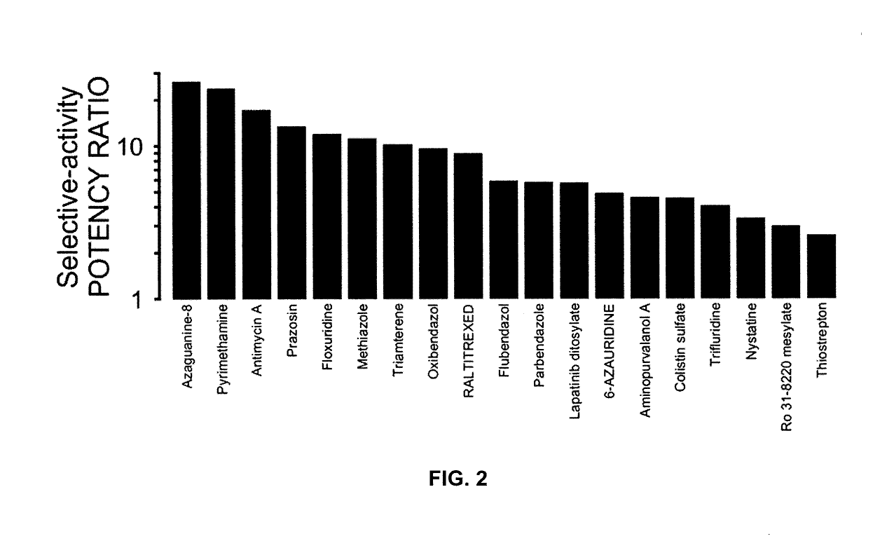

It has surprisingly been found that the compounds listed in FIG. 2 are selective for cancer stem cells relative to normal stem cells. As shown in Example 1, these compounds have been shown to have a Selectivity activity ratios [EC50 (v1O4)/EC50 (H9)] greater than 3 and are therefore preferentially targeting variant neoplastic stem cells relative to normal stem cells. Furthermore, as set out in Example 4, the polyene macrolide Nystatin was more effective than cytarabine (AraC) in a methylcellulose assay which provides a functional and quantitative measure of stem cell proliferation/clonogenic potential based on the formation of colony forming units in vitro. Other polyene macrolides that share structural and functional features with Nystatin such as amphotericin B, as well as analogs and pharmaceutically acceptable salts thereof, are also expected to preferentially target cancer stem cells and be useful for the treatment of cancer as described herein.

Accordingly, in one embodiment there is provided a method of inducing the differentiation of cancer stem cells comprising contacting the cancer stem cells with a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton. Also provided is a method of or reducing the proliferation of cancer stem cells comprising contacting the cancer stem cells with a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton. In one embodiment, there is also provided a method of inducing the differentiation and/or reducing the proliferation of cancer stem cells comprising contacting the cancer stem cells with a polyene macrolide. In one embodiment, the polyene macrolide is selected from Nystatin, amphotericin B, analogs thereof and pharmaceutically acceptable salts thereof. Optionally, the cancer stem cells are in vivo, in vitro or ex vivo.

Compounds identified according to the selective-activity assay described in Example 1 are expected to be useful for reducing the proliferation of cancer stem cells and therefore also useful for the treatment of cancer. Accordingly, in one embodiment there is provided is a method of treating cancer or a pre-cancerous disorder comprising administering to a subject a therapeutically effective amount of a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton. Also provided are uses of a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton for the treatment of cancer. In one embodiment, the compound is Triamterene, an analog thereof or pharmaceutically acceptable salt thereof. In one embodiment, the compound is colistin sulfate, an analog thereof pharmaceutically acceptable salt thereof. In one embodiment, the methods or uses described herein are useful to treat a precancerous disorder.

In one embodiment, there is provided a method of treating cancer or a pre-cancerous disorder comprising administering to a subject a therapeutically effective amount of a polyene macrolide. For example, in one embodiment the polyene macrolide is selected from nystatin and amphotericin B. In one embodiment, the polyene macrolide is an analog or pharmaceutically acceptable salt of nystatin or amphotericin B. In one embodiment, the cancer is leukemia, optionally acute myeloid leukemia or acute myelogenous leukemia (AML).

In one embodiment, the compounds described herein are prepared or formulated for administration to a subject in need thereof as known in the art. Conventional procedures and ingredients for the selection and preparation of suitable formulations are described, for example, in Remington's Pharmaceutical Sciences (2003−20th edition) and in The United States Pharmacopeia: The National Formulary (USP 24 NF19) published in 1999.

In one embodiment, the compounds described herein may be used or administered in a pharmaceutical composition comprising additional agents or compounds to e.g. stabilize the formulation or improve its characteristics for a particular purpose. For example, in one embodiment, the compound is a polyene macrolide such as nystatin or amphotericin B and the formulation comprises a surfactant or agent to encourage the solubility of the polyene macrolide and/or prevent or reduce the formation of micelles or aggregates. In one embodiment, the pharmaceutical compositions include a polyene macrolide and an FDA-approved surfactant such as Cremophor EL or Tween 80 that help solubilize polyene macrolides at higher concentrations (see e.g. Croy and Kwon, 2005).

Also disclosed herein is the use of a compound that selectively targets cancer stem cells as described herein for the manufacture of a medicament. In one embodiment, the medicament is for the treatment of a cancer and/or a precancerous disorder. In one embodiment, the medicament is for the differentiating and/or reducing the proliferation of cancer stem cells. In one embodiment, the medicament is for selectively killing cancer stem cells relative to normal stem cells. In one embodiment, the medicament is a pharmaceutical composition comprising a compound as described herein. In one embodiment, the medicament is for the treatment of leukemia, optionally AML.

The following non-limiting examples are illustrative of the present disclosure:

### EXAMPLES

### Example 1

### Identification and Characterization of Compounds that Selectively Target Cancer Stem Cells

The inventors have previously described a variant human pluripotent stem cell (hPSC) line that displays neoplastic features which include enhanced self-renewal and survival, along with aberrant block in terminal differentiation capacity in vitro and in vivo (Werbowetski-Ogilvie et al., 2009). Based on these similarities in functional properties to somatic CSCs, variant neoplastic stem cells are useful as a surrogate for somatic CSCs and are amenable for high content and high throughput screening in vitro. A screening platform was developed to identify small molecules that selectively target variant neoplastic stem cells whilst having little effect on normal hPSCs. This differential screening platform is capable of identifying potent candidate drugs that selectively target somatic CSCs while sparing healthy SC capacity.

Oct4 provides a reliable indicator of loss of self-renewing pluripotent state and differentiation induction of normal and neoplastic hPSCs. To provide a more straightforward method for detecting loss of Oct4 during induced differentiation of neoplastic hPSCs, GFP-reporter lines were generated by transduction of neoplastic hPSCs with the EOS-GFP reporter (v1H9-Oct4-GFP) (Hotta et al., 2009). GFP intensity was observed to be correlated with Oct4 expression in treatments that favored self-renewal stability and conditions that induce differentiation with the addition of BMP4. This response was consistently found using an additional neoplastic hPSC line, v2H9 (Werbowetski-Ogilvie et al., 2009) transduced with the same EOSlentivirus GFP-reporter (v2H9-Oct4-GFP). However, many common methods of detecting Oct4 are available including immunohistochemistry and other reporter systems, each of which can be used to in the assay described herein.

### Screening Assay for Selective Anti-Cancer Stem Cell Compounds

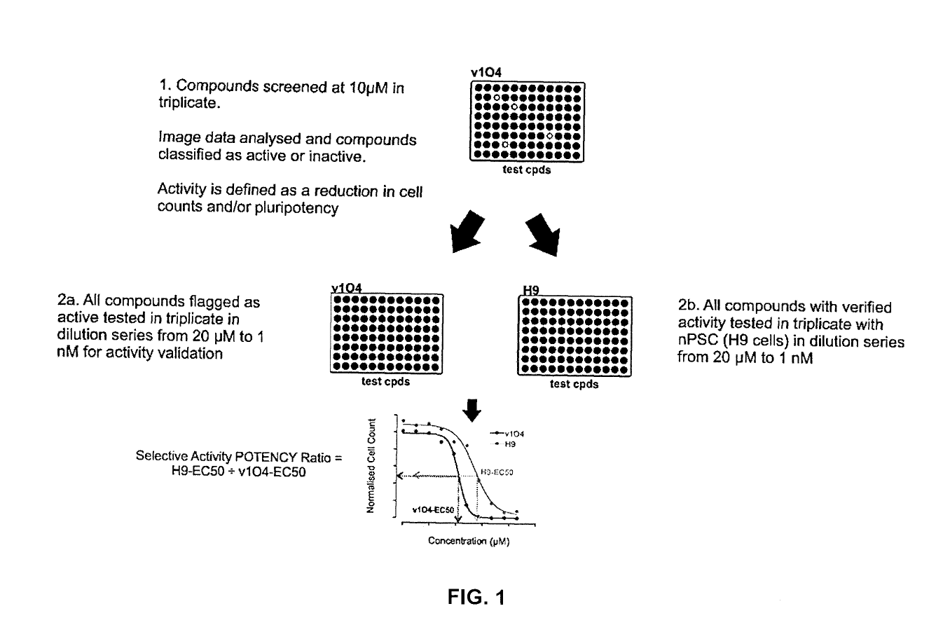

The compounds described herein were identified using the screening assay shown in FIG. 1. This screening assay improves upon previous screening procedures described by the inventors (Sachlos et al., 2012, incorporated by reference herein in its entirety).

In a first stage shown in FIG. 1, variant neoplastic stem cells (v1O4 cells, also known as v1H9-Oct4-GFP cells) were treated with different chemical libraries to identify active compounds or ‘hits’. Compounds were classified as hits if they induced a loss of pluripotency (LOP, a measure based on detection of a reporter of Oct 4 levels, which in this instance is a GFP signal output) and a reduction in cell counts (below 750 cells per acquired image). Compounds that reduced cell counts below 100 were classified as highly toxic and not considered as useful.

Briefly, variant neoplastic stem cells (v1O4) cells were seeded into Matrigel-coated 96 well plates (5000 cells/well) containing mouse embryonic fibroblast conditioned media (MEFCM) supplemented with 8 ng/mL bFGF, and treated for 72 hours with compounds dissolved in DMSO. The final concentration of each compound used in treatment was either 10 μM or 1 μM (n=3). Control wells were treated with 0.1% DMSO (low control) or 100 ng/ml BMP4 (high control to induce LOP). At the end of 72 hours, cells were fixed, stained with Hoechst and imaged by automated microscopy. GFP intensity and Hoechst signal were quantified as measures of LOP and cell count, respectively, and compounds with a Z-score of more than 3 standard deviations from the mean for reduced cell count and LOP were chosen as hits. Compounds identified as hits were then validated in the second stage of the assay shown in FIG. 1.

The second stage of the assay represents an improvement over previous quantitative flow-cytometry methods for determining compound potency and detecting differences in response between variant neoplastic stem cells and normal stem cells. In the second stage also shown in FIG. 1, 8- or 10-point dilutions for each compound were tested on variant neoplastic stem cells (v1O4) and normal stem cells (H9 cells) cells to generate dose-response curves. For each compound, the effective concentration values for 50% reduction in cell counts (EC50) were extrapolated from the dose-response curves from v1O4 and H9 treated cells. Dose response data were fit with a 4-parameter Hill equation to derive EC50, slopes, min and max values using IDBS ActivityBase software. The EC50 values were then used to calculate a selective-activity potency ratio (H9 EC50/v1O4 EC50). A ratio value above 1 indicates the compound is more potent against v1O4 cells than against H9 cells. The ratio values were then used as a basis for identifying high selective-activity compounds that could potentially induce differentiation or cell death of cancer stem cells but not normal stem cells. Testing a compound on the variant neoplastic stem cells and the normal stem cells at a number of different concentrations allows for the generation of dose response curves and the identification of compounds which exhibit selective activity that may not be identified by screening at only a single concentration or over a limited range of concentrations.

### Identification of Anti-Cancer Stem Cell Compounds

Selectivity activity ratios [EC50 (v1O4)/EC50 (H9)] were calculated as discussed above for a number of compounds screened using the assay shown in FIG. 1. A ratio value of 3 was selected as a threshold for identifying high selective-activity compounds. These compounds are expected to selectively induce differentiation/toxicity in cancer stem cells but have minimal effects on normal stem cells. The compounds identified using the screening assay with the highest selective-activity ratio are shown in FIG. 2. These compounds include Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton.

### Analysis of Dose-Response Curves Permits the Identification of Anti-Cancer Stem Cell Agents

Most primary high-throughput screening methods use a single concentration point to interrogate the response of an assay due to treatment with a compound. In contrast, the compounds disclosed herein were identified using assays and conditions for manipulating variant neoplastic stem cells and normal stem cells at a plurality of concentrations in order to generate dose-response curves and identify and validate compounds as selective for cancer stem cells. Data from these curves were used to distinguish differences in responses of variant neoplastic stem cells and normal stem cells to compounds with varying potencies.

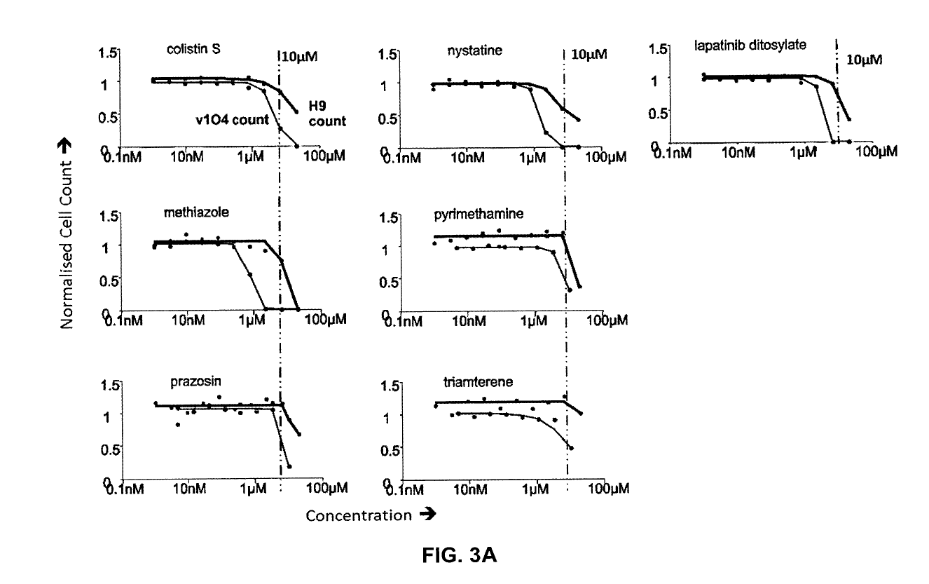

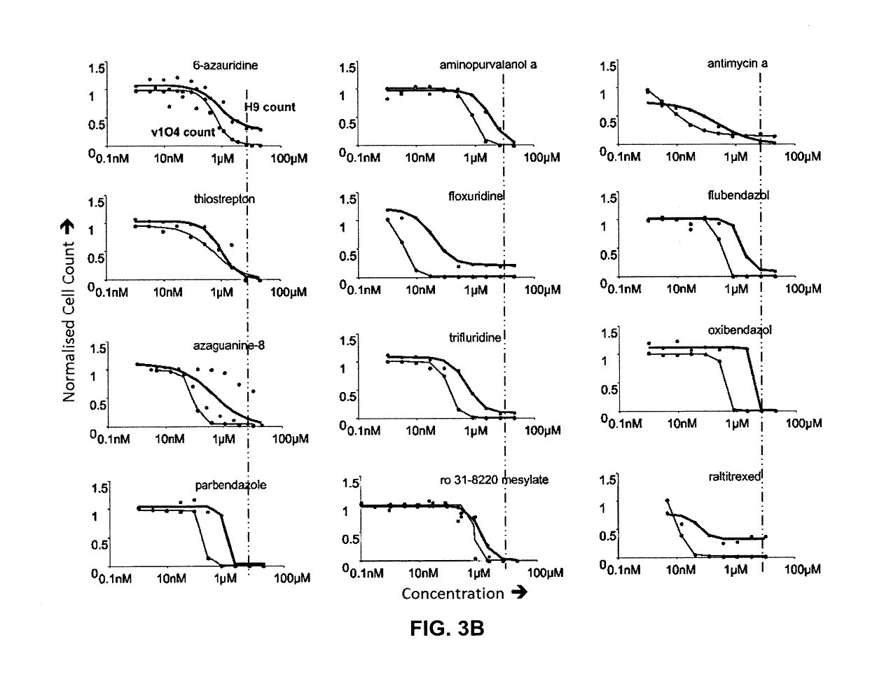

FIG. 3A shows 7 selective-active compounds that could have been identified by testing the cells at a single 10 μM concentration point. FIG. 3B shows the dose-response curves for the other 12 selective-active compounds. Based on a single 10 μM concentration point, many of these compounds would have not have been considered selective for cancer stem cells, such as 8-azaguanine, parbendazole or 31-8220.

### Example 2

### Anti-Cancer Compounds are Rarely Anti-Cancer Stem Cell Compounds

The chemical libraries used for in the screening assays described herein contained compounds that are described as known or current anti-cancer therapeutics. Many of these anti-cancer therapeutics presumably have shown toxicity against cancer cell lines.

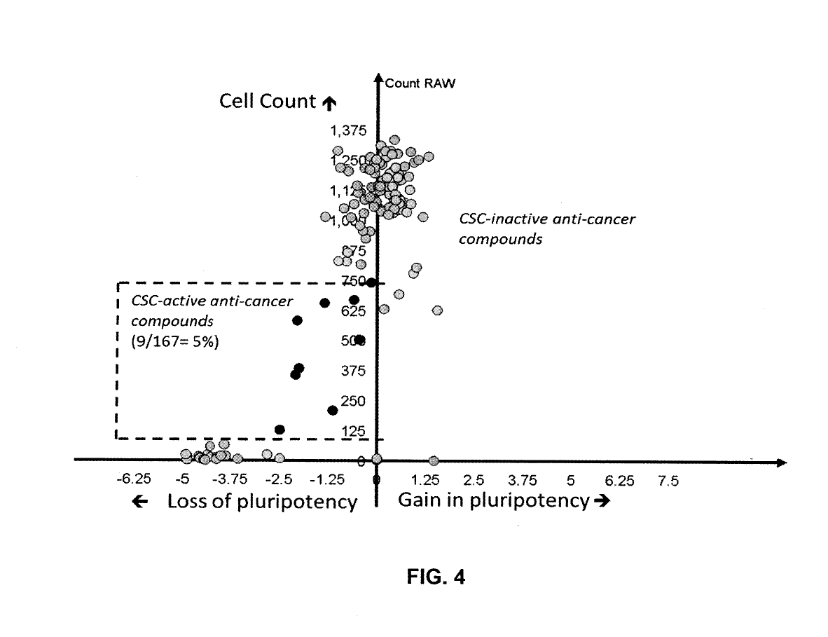

A MetaDrug search was performed for small molecule drugs with available structures that are used in treatment of human cancers (‘neoplasms’). This search found 167 such anti-cancer compounds from the combined NIH, PWK, TOCRIS and CCC libraries. These anti-cancer compounds were plotted as shown in FIG. 4 and only a small subset of them (5%) were identified as having activity against variant neoplastic stem cells (v1O4 cells). This suggests that the screening assay described herein is highly stringent or is identifying anti-cancer compounds in a unique manner. Furthermore, compounds previously identified as anti-cancer compounds are unlikely to be specific anti-cancer stem cell agents.

### Example 3

### Some High Selective-Activity Compounds have Low p53 Stress Response Activation Activity

AML is characterized by neoplastic hematopoietic cells that are blocked in their ability to differentiate into mature cells. Similarly, variant neoplastic stem cells are also refractory to normal differentiation cues (See Werbowetski-Ogilive et at, 2009). Agents that can induce differentiation of neoplastic progenitor/stem cells represent a promising strategy for the treatment of certain cancers. Treatment of acute promyelocytic leukemia (APL) using all-trans retinoic acid (ATRA) and arsenic trioxide are exemplary applications of this strategy. These compounds are thought to eradicate the cancer stem cells that maintain the cancer by inducing differentiation.

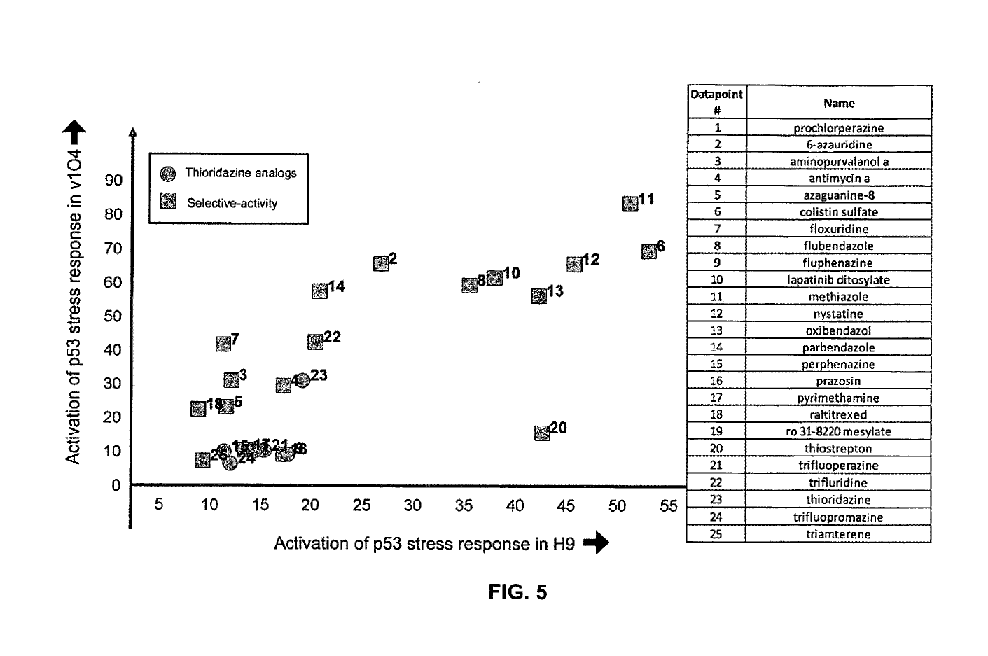

To identify compounds demonstrated to have high-selectivity shown in FIG. 2 that are also efficient in inducing differentiation, treated variant neoplastic stem cells were analyzed for changes in p53-dependent cytotoxic stress response. Variant neoplastic stem cells (v1O4 cells) and normal H9 stem cells were fixed and stained for p53 expression following treatment with selective-activity compounds. The percentage of v1O4 and H9 cells staining positive for p53 were then plotted for each compound as shown in FIG. 5. High levels of p53 activation indicated high cellular toxicity. Although selective-activity compounds caused varying levels of p53 activation in both v1O4 and H9 cells, the v1O4 cells generally appeared more sensitive relative to normal H9 cells.

High selective-activity compounds clustered near the bottom left corner did not significantly increase the p53-dependent stress response in v1O4 and H9 cells. This group may contain potential candidates for compounds that selectively differentiate v1O4 cells. p53 levels of variant neoplastic stem cells treated with thioridazine and thioridazine-analogs were determined and are also shown on FIG. 5 as black dots. As shown in FIG. 5, the thioridazine analogs appeared in this same bottom left corner. Thioridazine is known to act on variant neoplastic stem cells by inducing differentiation through a loss of pluripotency (See Sachlos et al., 2012).

### Example 4

### Nystatin Targets the Cancer Stem Cell Fraction of a Primary Cancer Sample without Affecting Normal Human Progenitor/Stem Cell Proliferation

Several of the compounds identified in Example 1 as having anti-cancer stem cell activity are not typically described as anti-cancer agents, including the approved antifungal drug, Nystatin (FIGS. 2 and 3A). Nystatin is a polyene macrolide that acts by binding to membrane sterols such as ergosterol, which results in increased permeability of fungal cell membranes. This compound was chosen to undergo further in vitro testing to validate its anti-cancer stem cell effects in a primary human cancer, in this case acute myeloid leukemia (AML).

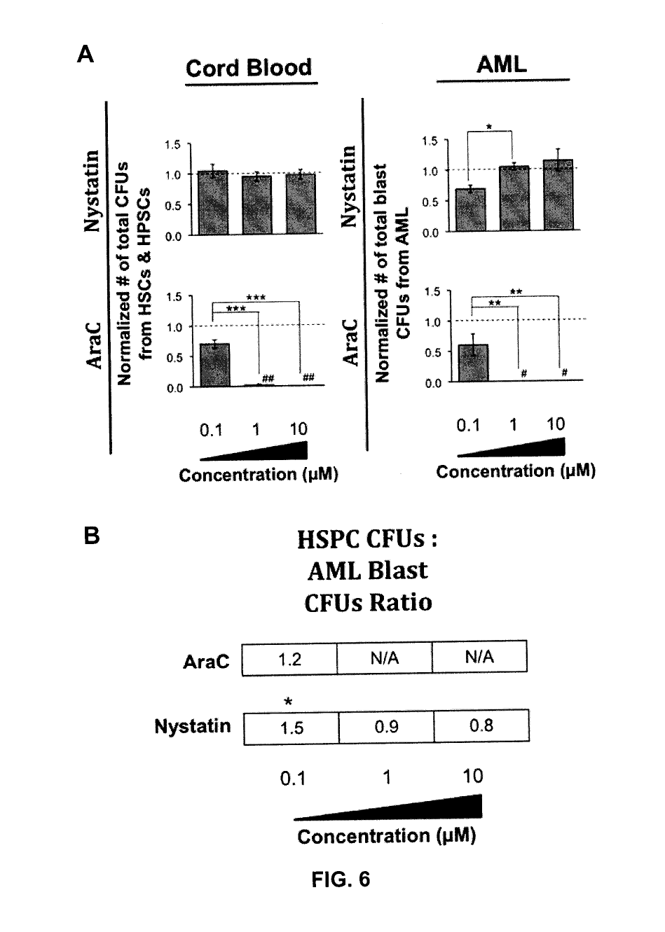

Methylcellulose assays provide a functional and quantitative measure of hematopoietic progenitor/stem cell proliferation/clonogenic potential based on the formation of colony-forming units (CFUs) in vitro. Hematopoietic progenitor/stem cells from lineage-depleted human umbilical cord blood are capable of proliferation and differentiation to all blood lineages. AML blast cells from leukemia patients are myeloid progenitor cells blocked in differentiation that are sustained by a self-renewing leukemic stem cell(s). In this experiment, lineage-depleted cord blood cells and AML samples were each treated with Nystatin for one day and then cultured in methylcellulose for 14 days after which the numbers of CFUs were determined. At a concentration of 0.1 μM, Nystatin treatment reduced the ability of AML cells to form CFUs while having little effect on normal hematopoietic progenitor/stem cell proliferation activity (FIG. 6A, top row).

Methylcellulose assays were also performed on cord blood and AML cells treated with cytarabine (AraC)—a front-line chemotherapy used in the treatment of AML—at similar concentrations (FIG. 6A, bottom row). Like Nystatin, AraC treatment at 0.1 μM reduced the ability of AML samples to form CFUs. However, the same treatment also affected the CFU-forming potential of cord blood cells. Higher concentrations of AraC proved toxic to both cell types. Treatment of AML cells with higher concentrations of Nystatin (1 and 10 μM) unexpectedly had no effect on their CFU forming potential (FIG. 6A, top row). Increasing concentrations of Nystatin were expected to result in further reductions in AML CFU-forming ability and possibly cord blood CFU-forming ability as well. Polyene macrolide antifungals such as Nystatin are amphipathic and can exist as monomers in solution at low concentrations and also as micelles or even aggregates at higher concentrations (>1 μM) (Castanho et al., 1992). One explanation for the effects seen with higher concentrations of Nystatin was that micelles or aggregates are present at the higher test concentrations and this somehow interfered with their ability to specifically inhibit AML cell proliferation. Optionally, the polyene macrolides described herein may be used in a pharmaceutical formulation also comprising a surfactant or other agent in order to prevent or minimize micelle and/or aggregate formation. For example, the polyene macrolides could be solubilized using FDA-approved surfactants such as Cremophor EL or Tween 80 that help solubilize these anti-fungals at higher concentrations (see e.g. Croy and Kwon, 2005).

The ratio of total CFUs generated from cord blood cells versus blast CFUs generated from AML samples was calculated to quantify the selectivity of Nystatin in targeting AML cells (FIG. 6B). A ratio greater than 1 would indicate that this compound selectively reduces the potential of AML cells to form colonies. Nystatin at 0.1 μM yielded a selectivity ratio of 1.5, showing significantly lower number of AML blast-CFUs relative to the number of normal cord blood CFUs (FIG. 6B, P=0.03 t-test for cord blood vs. AML with nystatin treatment at 0.1 μM). This value was higher than the selectivity ratio calculated for AraC at the 0.1 μM, which showed no statistically significant difference in reduction of the CFU-forming ability of treated cord blood versus AML samples. Other polyene macrolides such as amphotericin B or Nystatin analogs such as those described by Brautaset et al., 2008 are expected to exhibit similar specific anti-proliferative activity against AML cells.

Methylcellulose Assay.

Cord blood and AML patient cells were seeded in 96-well plates at 500 cells and 25000 cells respectively in 50 μL medium containing StemSpan™ (Stemcell Technologies), 200 ng/mL stem cell factor (SCF, R&D Systems), 200 ng/mL Flt-3 (R&D Systems) and 40 ng/mL thrombopoietin (TPO, Stemcell Technologies). Then, 50 μL StemSpan containing 0.2 μM, 2 μM or 20 μM compounds was added to each well containing the cells. The cells were incubated for 24 hours at 37° C., after which the compound medium was removed from the wells and replaced with 50 μL medium containing StemSpan, 100 ng/mL SCF, 100 ng/mL Flt-3 and 20 ng/mL TPO. The cells from each well were mixed with 500 μL Methocult™ (Stemcell Technologies, H4434), and seeded into 12-well plates. The samples were imaged and scored after 14 days of incubation at 37° C.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Brautaset, T., Sletta, H. et al (2008) Improved Antifungal Polyene Macrolides via Engineering of the Nystatin Biosynthetic Genes in Strepomyces noursei. Chemistry and Biology 15, 1198-1206.
Castanho M., Coutinho, A. et al (1992) Absorption and Fluorescence Spectra of Polyene Antibiotics in the Presence of Cholesterol. J. of Biol. Chem. 267(1), 204-209.
Chen Y., Wang, S. et al (2011) Cholesterol sequestration by nystatin enhances the uptake and activity of endostatin in endothelium via regulating distinct endocytic pathways. Blood 117, 6392-6403.
Croy, S R., Kwon, G S. (2005) Polysorbate 80 and Cremophor EL micelles deaggregate and solubilize nystatin at the core-corona interface. J of Pharm Sci 94, 2345-2354.
Dick, J. E. (2009). Looking ahead in cancer stem cell research. Nat Biotechnol 27, 44-46.
Frese, K. K., and Tuveson, D. A. (2007). Maximizing mouse cancer models. Nat Rev Cancer 7, 645-658.
Guan, Y., Gerhard, B., and Hogge, D. E. (2003). Detection, isolation, and stimulation of quiescent primitive leukemic progenitor cells from patients with acute myeloid leukemia (AML). Blood 101, 3142-3149.
Gupta, P. B., Onder, T. T., Jiang, G., Tao, K., Kuperwasser, C., Weinberg, R. A., and Lander, E. S. (2009). Identification of selective inhibitors of cancer stem cells by high-throughput screening. Cell 138, 645-659.
Hotta, A., Cheung, A. Y., Farra, N., Vijayaragavan, K., Seguin, C. A., Draper, J. S., Pasceri, P., Maksakova, I. A., Mager, D. L., Rossant, J., et al. (2009). Isolation of human iPS cells using EOS lentiviral vectors to select for pluripotency. Nat Methods 6, 370-376.
Jordan, C. T. (2009). Cancer stem cells: controversial or just misunderstood? Cell Stem Cell 4, 203-205.
Li, X., Lewis, M. T., Huang, J., Gutierrez, C., Osborne, C. K., Wu, M. F., Hilsenbeck, S. G., Pavlick, A., Zhang, X., Chamness, G. C., et al. (2008). Intrinsic resistance of tumorigenic breast cancer cells to chemotherapy. J Natl Cancer Inst 100, 672-679.
Raj, L., Ide, T., Gurkar, A. U., Foley, M., Schenone, M., Li, X., Tolliday, N. J., Golub, T. R., Carr, S. A., Shamji, A. F., et al. (2011). Selective killing of cancer cells by a small molecule targeting the stress response to ROS. Nature 475, 231-234.
Sachlos E, Risueño R M, Laronde S, Shapovalova Z, Lee J H, Russell J, Malig M, McNicol J D, Fiebig-Comyn A, Graham M, Levadoux-Martin M, Lee J B, Giacomelli A O, Hassell J A, Fischer-Russell D, Trus M R, Foley R, Leber B, Xenocostas A, Brown E D, Collins T J, Bhatia M. Identification of drugs including a dopamine receptor antagonist that selectively target cancer stem cells. Cell. 2012 Jun. 8; 149(6):1284-97. Epub 2012 May 24.
Shoemaker, R. H. (2006). The NCI60 human tumour cell line anticancer drug screen. Nat Rev Cancer 6, 813-823.
Smith, T. J., Khatcheressian, J., Lyman, G. H., Ozer, H., Armitage, J. O., Balducci, L., Bennett, C. L., Cantor, S. B., Crawford, J., Cross, S. J., et al. (2006). 2006 update of recommendations for the use of white blood cell growth factors: an evidence-based clinical practice guideline. J Clin Oncol 24, 3187-3205.
Werbowetski-Ogilvie, T. E., Bosse, M., Stewart, M., Schnerch, A., Ramos-Mejia, V., Rouleau, A., Wynder, T., Smith, M. J., Dingwall, S., Carter, T., et al. (2009). Characterization of human embryonic stem cells with features of neoplastic progression. Nat Biotechnol 27, 91-97.

1.-10. (canceled)

11. A method of preferentially inducing the differentiation of cancer stem cells or reducing the proliferation of cancer stem cells comprising contacting the cancer stem cells with a polyene macrolide.

12. The method of claim 11, wherein the polyene macrolide is selected from Nystatin, Amphotericin B, analogs thereof and pharmaceutically acceptable salts thereof.

13. The method of claim 12, wherein the polyene macrolide is Nystatin or a pharmaceutically acceptable salt thereof.

14. The method of claim 11, wherein the polyene macrolide preferentially induces the differentiation of cancer stem cells relative to normal stem cells, preferentially reduces the proliferation of cancer stem cells relative to normal stem cells or preferentially kills cancer stem cells relative to normal stem cells.

15. (canceled)

16. (canceled)

17. The method of claim 14, wherein the normal stem cells are H9 cells, hematopoietic stem cells or hematopoietic progenitor cells.

18. (canceled)

19. The method of claim 11, wherein the cancer stem cells are in vitro, in vivo or ex vivo.

20. The method of claim 11, wherein the cancer stem cells are leukemic cancer stem cells.

21. A method of treating cancer in a subject in need thereof, comprising administering to the subject a therapeutically effective amount of a polyene macrolide.

22. The method of claim 21, wherein the polyene macrolide is selected from Nystatin, Amphotericin B, analogs thereof and pharmaceutically acceptable salts thereof.

23. The method of claim 22, wherein the polyene macrolide is Nystatin or a pharmaceutically acceptable salt thereof.

24. The method of claim 21, wherein the subject has leukemia or is suspected of having leukemia.

25. The method of claim 24, wherein the leukemia is acute myeloid leukemia (AML).

26. The method of claim 21, wherein the polyene macrolide preferentially induces the differentiation of cancer stem cells relative to normal stem cells, reduces the proliferation of cancer stem cells relative to normal stem cells, or preferentially kills cancer stem cells relative to normal stem cells.

27. (canceled)

28. The method of claim 26, wherein the normal stem cells are H9 cells, hematopoietic stem cells or hematopoietic progenitor cells.

29. (canceled)

30. The method of claim 21, wherein the polyene macrolide is in a pharmaceutical composition comprising a surfactant.

31. The method of claim 21, wherein the subject is in remission.

32. A method of preferentially inducing the differentiation of cancer stem cells or reducing the proliferation of cancer stem cells comprising contacting the cancer stem cells with a compound selected from Azaguanine-8, Pyrimethamine, Antimycin A, Prazosin, Floxuridine, Methiazole, Triamterene, Oxibendazol, Raltitrexed, Flubendazol, Parbendazole, Lapatinib ditosylate, 6-Azauridine, Aminopurvalanol A, Colistin sulfate, Trifuridine, Nystatin, Ro 31-8220 mesylate and Thiostrepton.

33. The method of claim 32, wherein the compound preferentially induces the differentiation of cancer stem cells relative to normal stem cells, preferentially reduces the proliferation of cancer stem cells relative to normal stem cells or preferentially kills cancer stem cells relative to normal stem cells.

34. (canceled)

35. The method of claim 32, wherein the normal stem cells are H9 cells, hematopoietic stem cells or hematopoietic progenitor cells.

36. The method of claim 32, wherein the cancer stem cells are in vitro, in vivo or ex vivo.

37. The method of claim 32, wherein the cancer stem cells are leukemic cancer stem cells.

38. The method of claim 32, wherein the compound is Triamterene.

39. The method of claim 32, wherein the compound is Colistin sulfate.

40-46. (canceled)
